(19) United States
(12) Patent Application Publication
(10) Pub. No.: US 2021/0401769 A1
(43) Pub. Date: Dec. 30, 2021

(54) TREATMENT OF FRAGILE X SYNDROME WITH CANNABIDIOL

(71) Applicant: ZYNERBA PHARMACEUTICALS, INC., Devon, PA (US)

(72) Inventors: James Griesser, Havertown, PA (US); Thomas W. Dobbins, Blue Bell, PA (US); Terri Sebree, Gladwyne, PA (US)

(73) Assignee: ZYNERBA PHARMACEUTICALS, INC., Devon, PA (US)

(21) Appl. No.: 17/360,781

(22) Filed: Jun. 28, 2021

Related U.S. Application Data
(60) Provisional application No. 63/045,664, filed on Jun. 29, 2020.

(51) Int. Cl. A61K 31/05; A61K 9/00; A61K 9/06; A61P 25/18

(52) U.S. Cl. CPC A61K 31/05; A61P 25/18; A61K 9/06; A61K 9/0014

(57) ABSTRACT

The present technology relates to a method of treating one or more symptoms of Full Methylation (Fmet) Fragile X Syndrome in a subject that includes administering an effective amount of cannabidiol to the subject such that one or more symptoms of Fragile X Syndrome are treated.

### CROSS-REFERENCE TO RELATED APPLICATIONS

This application claims benefit and priority to U.S. Provisional Application No. 63/045,664, filed Jun. 29, 2020, entitled “Treatment of Fragile X Syndrome with Cannabidiol.” The content of which is incorporated herein by reference in its entirety.

### FIELD OF THE TECHNOLOGY

The present disclosure relates to methods of treating one or more behavioral symptoms of Fragile X Syndrome in a subject having full methylation of the FMR1 gene (Fmet) by administering an effective amount of cannabidiol (CBD) to the subject.

### BACKGROUND

Cannabinoids are a class of chemical compounds found in the Cannabis plant. The two primary cannabinoids contained in Cannabis are cannabidiol, or CBD, and 49-tetrahydrocannabinol, or THC. CBD lacks the psychoactive effects of THC. Studies have shown that CBD can be used to treat disorders such as epilepsy, arthritis, and cancer.

FXS is the most common inherited intellectual disability in males and a significant cause of intellectual disability in females. It is caused by a mutation in the Fragile X Mental Retardation 1 (FMR1) gene located on the X chromosome and leads to dysregulation of the endocannabinoid system including reductions in endogenous cannabinoids (2-AG and anandamide [AEA]). The disorder negatively affects synaptic function, plasticity and neuronal connections, and results in a spectrum of intellectual disabilities, social anxiety, and memory problems. In the US, there are about 71,000 patients suffering with FXS.

“Behavior problems are often the most significant concern reported by parents, and high levels of stress and depression and low levels of quality of life for parents are commonly associated with elevated problem behaviors in children.” Wheeler A, Raspa M, Bann C, Bishop E, Hassl D, Sacco H, Bailey D B. 2014. Anxiety attention problems, hyperactivity, and the Aberrant Behavior Checklist in fragile X syndrome. Am J Med Genet Part A 164A:141-155, 141. “As a result, reduction in behavior problems is a primary focus of ongoing clinical trials testing the efficacy of new medications for FXS.” Wheeler at pages 141-142.

The Anxiety, Depression, and Mood Scale (ADAMS) is an instrument that is used by clinicians, doctors, and researchers to assess the level of anxiety, depression and mood in patients with intellectual disabilities, including FXS. ADAMS consists of questions grouped into five subscales, including (i) general anxiety, (ii) social avoidance, (iii) compulsive behavior, (iv) manic/hyperactive behavior, and (v) depressed mood. Each question is answered by a clinician/doctor on a four-point scale ranging from 0 (“not a problem”) to 3 (“severe problem”). In addition to subscale scores, the ADAMS yields a total score.

The original Aberrant Behavior Checklist (ABC) was “designed to assess behavioral concerns of adults within institutional settings.” Wheeler at page 142. Since then, the original ABC has been adapted to address patients who are not institutionalized and specifically to address FXS. Id. The Aberrant Behavior Checklist—FXS Specific (ABC-FXS) scale is used by clinicians, doctors, and researchers to access certain behaviors in patients with FXS. The ABC-FXS scale has six subscales including (i) irritability, (ii) hyperactivity, (iii) socially unresponsive/lethargic, (iv) social avoidance, (v) stereotypy, and (vi) in appropriate speech. Similar to ADAMS, the ABC-FXS scale is a four-point Likert-type scale ranging from 0 (not a problem) to 3 (problem is severe).

### SUMMARY

The present disclosure relates to a method of treating one or more behavioral symptoms of Fragile X Syndrome in a subject having full methylation of the FMR1 gene, the method. The method includes transdermally administering an effective amount of cannabidiol (CBD) to the subject.

In some embodiments, one or more symptoms is a behavioral symptom of Fragile X Syndrome. In some embodiments, the behavioral symptom is social avoidance. In some embodiment, treating includes improvement in clinical global impression (CGI-I).

In some embodiments, the CBD is (−)-CBD. The effective amount of CBD can be between about 50 mg to about 500 mg daily. In some embodiments, the effective amount of CBD is initiated at about 50 mg daily and titrated up to about 500 mg daily. The effective amount of CBD can be initiated at about 50 mg daily and titrated up to about 250 mg daily. In some embodiments, the effective amount of CBD is initiated at 250 mg daily. The effective amount of CBD can be initiated at 500 mg daily. In some embodiments, the 500 mg daily dose is administered to patients that weigh greater than 35 kg. The CBD can be administered in a single daily dose or in two daily doses. In some embodiments, the effective amount of CBD can be 390 mg in divided daily doses.

In some embodiments, the total daily dose of CBD is 250 mg. In some embodiments, the total daily dose of CBD is 500 mg. In some embodiments, the total daily dose of CBD is 250 mg or 500 mg. The dosing, in some embodiments, is weight-based. In some embodiments, the CBD is administered in a single daily dose. In some embodiments, the CBD is administered in two daily doses.

The CBD can be formulated as a gel or an oil. In some embodiments, the CBD is formulated as a gel, e.g., a permeation-enhanced gel. The gel can contain between 1% (wt/wt) CBD to 7.5% (wt/wt) CBD. In some embodiments, the gel contains 4.2% (wt/wt) CBD. In some embodiments, the gel contains 7.5% (wt/wt) CBD.

In some embodiments, the transdermal preparation can be a cream, a salve or an ointment. The CBD can be delivered by a bandage, pad or patch.

The CBD can be administered transdermally on the subject's upper arm and shoulder. In some embodiments, the CBD is administered transdermally on the subject's thigh or back. In some embodiments, the CBD is transdermally administered by application to the subject's arm.

The CBD can be synthetic CBD. The CBD can be purified CBD. The CBD can be botanically derived. In some embodiments, the CBD is not botanically derived.

Transdermally administering an effective amount of cannabidiol (CBD) can reduce an intensity of at least one adverse event or side effect relative to orally administering CBD. The at least one adverse event or side effect can be a gastrointestinal (GI) adverse event. The at least one adverse event or side effect can be liver function. In some embodiments, the at least one adverse event is somnolence. In some embodiments, the frequency and intensity of somnolence is reduced as an adverse event. In some embodiments, psychoactive effects are reduced or eliminated.

### DETAILED DESCRIPTION

As used herein, the term “treating” or “treatment” refers to mitigating, improving, relieving, or alleviating at least one symptom (such as a behavioral symptom) of a condition, disease or disorder in a subject, such as a human, or the improvement of an ascertainable measurement associated with a condition, disease or disorder.

As used herein, the term “clinical efficacy” refers to the ability to produce a desired effect in humans as shown through a Food and Drug Administration (FDA), or any foreign counterparts, clinical trial.

As used herein, the term “cannabidiol” or “CBD” refers to cannabidiol; cannabidiol prodrugs; pharmaceutically acceptable derivatives of cannabidiol, including pharmaceutically acceptable salts of cannabidiol, cannabidiol prodrugs, and cannabidiol derivatives. CBD includes, 2-[3-methyl-6-(1-methylethenyl)-2-cyclohexen-1-yl]-5-pentyl-1,3-benzenediol as well as to pharmaceutically acceptable salts, solvates, metabolites (e.g., cutaneous metabolites), and metabolic precursors thereof. The synthesis of CBD is described, for example, in Petilka et al., Helv. Chim. Acta, 52:1102 (1969) and in Mechoulam et al., J. Am. Chem. Soc., 87:3273 (1965), which are hereby incorporated by reference.

As used herein, the term “transdermally administering” refers to contacting the CBD with the patient's or subject's skin under conditions effective for the CBD to penetrate the skin.

### Fragile X Syndrome

Fragile X syndrome is a rare genetic developmental disability that is the leading known cause of both inherited intellectual disability and autism spectrum disorder, affecting 1 in 3,600 to 4,000 males and 1 in 4,000 to 6,000 females. It is the most common inherited intellectual disability in males and a significant cause of intellectual disability in females, and the leading genetic cause of autism spectrum disorder (ASD). The disorder negatively affects synaptic function, plasticity and neuronal connections, and results in a spectrum of intellectual disabilities and behavioral symptoms, such as social avoidance and irritability. In the US, there are about 71,000 people suffering with FXS, approximately 60% of whom have full methylation of the FMR1 gene.

FXS is caused by a mutation in FMR1, a gene which modulates a number of systems, including important effects on the endocannabinoid system, and most critically, codes for a protein called FMRP. This protein helps regulate the production of other proteins and plays a role in the development of synapses, which are critical for relaying nerve impulses, and in regulating synaptic plasticity. The FMR1 mutation manifests as multiple repeats of a DNA segment, known as the CGG triplet repeat. In most neurotypical people, the FMR1 gene correctly codes for the FMRP protein. In neurotypical individuals, there are CGG repeats, but these repeats only occur between 5 and 40 times. As a result, FMRP is manufactured at levels that enable control over behaviors like social avoidance and anxiety. In people with FXS, the CGG segment is repeated more than 200 times and in most cases causes the FMR1 gene to not function. However, the methylation of the FMR1 gene also plays a role in determining functionality of the gene. At greater than 90% methylation, which is considered “full methylation”, the FMR1 gene is silenced, therefore, no FMRP is produced, and the systems and processes that are expected to be affected by FMRP become dysregulated.

Patients with FXS can be classified as mosaic or non-mosaic. Mosaicism has been described as the coexistence of the full mutation and the pre-mutation CGG repeats in adjacent cells in the body. Patients with mosaic FXS may still produce some level of FMRP, as the FMR1 gene has not been fully silenced in all cells in the body. The amount of FMRP produced is generally modulated by methylation of the FMR1 gene. At greater than 90% methylation (considered full methylation), the FMR1 gene is considered fully silenced.

People with genetically confirmed full mutation Fragile X and full methylation of their FMR1 gene are generally the most severely impacted by the disorder.

The present disclosure relates to a method of treating one or more behavioral symptoms of fully methylated (Fmet) Fragile X Syndrome in a subject by transdermally administering an effective amount of cannabidiol (CBD) to the subject wherein one or more behavioral symptoms of Fragile X Syndrome are treated in the subject.

Clinical and preclinical data support the potential for CBD in treating epilepsy, arthritis, cancer, and Fragile X Syndrome. Therapeutic medicines have been developed that utilize innovative transdermal technologies to allow for sustained and controlled delivery of therapeutic levels of CBD. Transdermal delivery of cannabinoids (e.g., CBD) has benefits over oral dosing because it allows the drug to be absorbed through the skin directly into the bloodstream. This avoids first-pass liver metabolism, potentially enabling lower dosage levels of active pharmaceutical ingredients with a higher bioavailability and improved safety profile. Transdermal delivery also avoids the gastrointestinal tract, lessening the opportunity for GI related adverse events and the potential degradation of CBD by gastric acid into THC, which can be associated with unwanted psychoactive effects. Moreover, transdermal delivery of CBD reduces the intensity and frequency of somnolence adverse events, which are typically present in oral dosing of CBD. Transdermal delivery of CBD can avoid liver function adverse events, which are typically present in oral dosing of CBD. In some embodiments, transdermally administering an effective amount of CBD reduces an intensity of at least one adverse event by about 15% to about 95% relative to orally administering CBD.

The CBD can be in a gel form and can be pharmaceutically-produced as a clear, permeation-enhanced gel that is designed to provide controlled drug delivery transdermally with once- or twice-daily dosing. The CBD gel can between 1% (wt/wt) CBD to 7.5% (wt/wt) CBD. The CBD gel can have, for example, 4.2% (wt/wt) CBD or 7.5% (wt/wt) CBD). The CBD gel can be applied topically by the patient or caregiver to the patient's upper arm and shoulder, back, thigh, or any combination thereof.

The CBD gel can include diluents and carriers as well as other conventional excipients, such as wetting agents, preservatives, and suspending and dispersing agents.

The CBD gel can include a solubilizing agent, a permeation enhancer, a solubilizer, antioxidant, bulking agent, thickening agent, and/or a pH modifier. The composition of the CBD gel can be, for example, a. cannabidiol present in an amount of about 0.1% to about 20% (wt/wt) of the composition; b. a lower alcohol having between 1 and 6 carbon atoms present in an amount of about 15% to about 95% (wt/wt) of the composition; c. a first penetration enhancer present in an amount of about 0.1% to about 20% (wt/wt) of the composition; and d. water in a quantity sufficient for the composition to total 100% (wt/wt). Other formulations of the CBD gel can be found in International Publication No. WO 2010/127033, the entire contents of which are incorporated herein by reference.

### EXEMPLIFICATION

### Example 1: Treatment of FXS with CBD

A total of 20 patients (mean age=10.8, SD=4.0) were enrolled in a 12-week study. Eighteen patients (14 males, 4 females) aged 6 to 17 years of age (mean=11.2 SD=3.96) with Fragile X as confirmed by molecular documentation of FMR1 full mutation completed the open label FAB-C study through week 12. CBD gel was added on to other medications being administered. The first six weeks of the study were designed to titrate dosing in patients. Dosing was initiated at 50 mg CBD daily and could be increased to 250 mg CBD daily. Weeks 7 through 12 of the study comprised the maintenance period where patients were treated at the dose established by week six at a maximum of 250 mg CBD daily. At the completion of the study, patients could enter an open label extension study for up to 12 months.

The primary endpoint for the trial was the change in the total score of the Anxiety, Depression, and Mood Scale (ADAMS) from baseline to week 12. The ADAMS is a 28-item scale designed to assess general anxiety, social avoidance, compulsive behavior, manic/hyperactive behavior, and depressed mood. It has been validated in patients with FXS.

Results for the primary endpoint are summarized in Table 1, detailing efficacy scales mean (standard deviation) values at baseline and week 12 for the ADAMS Total Score.

TABLE 1

| | | | Percent Change | |
|---|---|---|---|---|
| | Baseline | Week 12 | from Baseline | |
| | (n = 18) | (n = 18) | (%; n = 18) | p Value* |
| ADAMS: Total | 32.1 | 18.1 | −43.61 | p <0.0001 |
| Score | (14.36) | (8.32) | | |

*P-values are presented for the comparison of the Week 12 value to the Baseline value for the total score and each subscale, among those who completed the study (n = 18).

The subscales of the ADAMS are summarized in Table 2, detailing efficacy scales mean (standard deviation) values at baseline and week 12.

TABLE 2

| | | | Percent Change | |
|---|---|---|---|---|
| | Baseline | Week 12 | from Baseline | |
| | (n = 18) | (n = 18) | (%; n = 18) | p Value* |
| ADAMS: Manic/ | 8.8 | 6.1 | −30.68 | p = 0.0003 |
| Hyperactive | (3.99) | (3.29) | | |
| Behavior Subscale | | | | |
| ADAMS: | 2.9 | 2.0 | −31.03 | p = 0.1417 |
| Depressed Mood | (3.94) | (2.35) | | |
| Subscale | | | | |
| ADAMS: Social | 9.9 | 4.8 | −51.52 | p = 0.0002 |
| Avoidance | (5.18) | (2.07) | | |
| Subscale | | | | |
| ADAMS: General | 9.4 | 4.6 | −51.06 | p < 0.0001 |
| Anxiety Subscale | (4.35) | (3.35) | | |
| ADAMS: | 2.7 | 1.4 | −48.15 | p = 0.0262 |
| Compulsive | (2.40) | (1.42) | | |
| Behavior Subscale | | | | |

*P-values are presented for the comparison of the Week 12 value to the Baseline value for the total score and each subscale, among those who completed the study (n = 18).

Compared to the baseline total score, the CBD transdermal gel treated patients has a 44% reduction (p<0.0001) in the ADAMS Total Score. Furthermore, the CBD transdermal gel treated patients has statistically and clinically significant improvement compared to baseline in all but one of the ADAMS subscales (i.e., manic/hyperactive behavior, social avoidance, general anxiety, and compulsive behavior) at week 12. A significant change was not observed for the depressed mood subscale of the ADAMS.

Multiple secondary efficacy endpoints including the Aberrant Behavior Checklist—FXS Specific (ABC-FXS), the Pediatric Anxiety Rating Scale (PARS-R), Visual Analog Scale (VAS) for Anxiety, Hyperactivity and Tantrum/Mood Lability, the Vineland Adaptive Behavior (VLD) III, and the Pediatric Quality of Life (PedsQL™). Both the PARS-R and the Vineland scales are clinician-rated, while the other scales are caregiver-rated.

The primary and secondary endpoints were evaluated prior to and following 12 weeks of drug administration. The results of the secondary endpoints reinforce the results demonstrated in the ADAMS. Consistent with findings from the ADAMS, patients taking the CBD transdermal gel demonstrated statistically and clinically significant 12-week reductions in all subscales of the ABC-FXS (i.e., irritability, hyperactivity, socially unresponsive/lethargic, social avoidance, stereotypy, and inappropriate speech), and both total score calculations of the PARS-R (i.e., 5- and 7-item).

Patients also showed significant improvement between Baseline and Week 12 scores for all remaining scales except for the Physical Function, School Functioning, and Social Functioning subscales of the PedsQL, as well as some subscales of the VLD (e.g., communication, daily living skills). Both the VLD and ADAMS are being administered in the extension Phase 2 of the trial.

Results from the ABC-FXS are summarized in Table 3, detailing efficacy scales mean (standard deviation) values at baseline and week 12.

TABLE 3

| | | | Percent | |
|---|---|---|---|---|
| | | | Change from | |
| | Baseline | Week 12 | Baseline | |
| | (n = 18) | (n = 18) | (%; n = 18) | p Value* |
| ABC: Irritability | 17.7 | 10.6 | −40.11 | p = 0.0096 |
| | (12.68) | (11.03) | | |
| ABC: Hyperactivity | 13.7 | 9.8 | −28.47 | p = 0.0237 |
| | (9.09) | (7.38) | | |
| ABC: Socially | 9.2 | 4.1 | −55.43 | p = 0.0034 |
| Unresponsive/Lethargic | (6.40) | (4.09) | | |
| ABC: Social | 5.1 | 2.3 | −54.90 | p = 0.0005 |
| Avoidance | (3.46) | (2.22) | | |
| ABC: Stereotypy | 8.1 | 3.2 | −60.49 | p = 0.0006 |
| | (5.91) | (3.07) | | |
| ABC: Inappropriate | 5.9 | 3.5 | −40.68 | p = 0.0018 |
| Speech | (2.30) | (2.66) | | |

*P-values are presented for the comparison of the Week 12 value to the Baseline, among those who completed the study (n = 18).

Results from the PARS-R are summarized in Table 4, detailing efficacy scales mean (standard deviation) values at baseline and week 12.

TABLE 4

| | | | Percent | |
|---|---|---|---|---|
| | | | Change from | |
| | Baseline | Week 12 | Baseline | |
| | (n = 18) | (n = 18) | (%; n = 18) | p Value* |
| PARS-R - 5 Item | 15.7 | 10.6 | −32.48 | p = 0.0006 |
| | (3.88) | (3.43) | | |
| PARS-R - 7 Item | 21.3 | 14.4 | −32.39 | p = 0.0004 |
| | (5.55) | (4.54) | | |

*P-values are presented for the comparison of the Week 12 value to the Baseline value, among those who completed the study (n = 18).

Results from the VAS for Anxiety, Hyperactivity and Tantrum/Mood Lability are summarized in Table 5.

TABLE 5

| | | | Percent | |
|---|---|---|---|---|
| | | | Change from | |
| | Baseline | Week 12 | Baseline | |
| | (n = 18) | (n = 18) | (%; n = 18) | p Value* |
| VAS- | 5.9 | 3.6 | −38.98 | p = 0.0002 |
| Hyperactivity/Impulsivity | (2.43) | (2.49) | | |
| VAS-Tantrum/Mood | 4.7 | 3.2 | −31.91 | p = 0.0023 |
| Liability | (2.09) | (2.18) | | |
| VAS-Anxiety | 6.0 | 3.8 | −36.67 | p = 0.0005 |
| | (2.05) | (1.93) | | |

*P-values are presented for the comparison of the Week 12 value to the Baseline value, among those who completed the study (n = 18).

Results from the PedsQL are summarized in Table 6, detailing efficacy scales mean (standard deviation) values at baseline and week 12.

TABLE 6

| | | | Percent | |
|---|---|---|---|---|
| | | | Change from | |
| | Baseline | Week 12 | Baseline | |
| | (n = 18) | (n = 18) | (%; n = 18) | p Value* |
| PedsQL: Total Score | 57.8 | 67.7 | 17.13 | p = 0.0100 |
| | (18.78) | (18.27) | | |
| PedsQL: Physical | 67.9 | 78.0 | 14.87 | p = 0.0606 |
| Functioning | (27.36) | (22.39) | | |
| PedsQL: Emotional | 64.0 | 78.3 | 22.34 | p = 0.0394 |
| Functioning | (20.72) | (16.63) | | |
| PedsQL: Social | 37.3 | 49.0 | 31.37 | p = 0.0717 |
| Functioning | (24.70) | (24.35) | | |
| PedsQL: School | 55.7 | 59.1 | 6.10 | p = 0.3580 |
| Functioning | (19.17) | (22.47) | | |
| PedsQL: Psychosocial | 52.4 | 62.2 | 18.70 | p = 0.0408 |
| Health | (17.22) | (18.91) | | |

*P-values are presented for the comparison of the Week 12 value to the Baseline value, among those who completed the study (n = 18).

Results from the VLD III are summarized in Table 7, detailing efficacy scales mean (standard deviation) values at baseline and week 12.

TABLE 7

| | | | Percent | |
|---|---|---|---|---|
| | | | Change from | |
| | Baseline | Week 12 | Baseline | |
| | (n = 18) | (n = 18) | (%; n = 18) | p Value* |
| VLD III: Overall | 46.1 | 48.9 | 6.07 | p = 0.0472 |
| Adaptive Behavior | (16.29) | (16.49) | | |
| Composite | | | | |
| VLD III: Communication | 36.7 | 39.2 | 6.81 | p = 0.2968 |
| | (18.52) | (20.34) | | |
| VLD III: Communication- | 3.9 | 5.3 | 35.90 | p = 0.0752 |
| Receptive | (3.43) | (4.34) | | |
| VLD III: Communication- | 3.3 | 3.7 | 12.12 | p = 0.5070 |
| Expressive | (3.63) | (4.07) | | |
| VLD III: Communication- | 4.4 | 3.8 | −13.64 | p = 0.0293 |
| Written | (3.81) | (3.64) | | |
| VLD III: Daily Living | 52.7 | 54.6 | 3.61 | p = 0.3911 |
| Skills | (21.19) | (18.46) | | |
| VLD III: Daily Living | 5.7 | 6.2 | 8.77 | p = 0.3374 |
| Skills-Personal | (4.26) | (4.33) | | |
| VLD III: Daily Living | 9.6 | 9.5 | −1.04 | p = 0.9395 |
| Skills-Domestic | (3.42) | (3.09) | | |
| VLD III: Daily Living | 4.6 | 4.7 | 2.17 | p = 0.5636 |
| Skills-Community | (3.09) | (2.93) | | |
| VLD III: Socialization | 45.9 | 50.9 | 10.89 | p = 0.0344 |
| | (16.22) | (17.83) | | |
| VLD III: Socialization- | 5.3 | 5.9 | 11.32 | p = 0.2937 |
| Interpersonal | (3.51) | (3.64) | | |
| Relationships | | | | |
| VLD III: Socialization- | 3.4 | 4.5 | 32.35 | p = 0.0350 |
| Play and Leisure | (2.91) | (3.93) | | |
| VLD III: Socialization- | 6.6 | 7.8 | 18.18 | p = 0.0246 |
| Coping Skills | (2.93) | (2.84) | | |
| VLD III: Maladaptive | 19.9 | 18.7 | −6.03 | p = 0.0486 |
| Behavior-Internalizing | (1.71) | (1.79) | | |
| VLD III: Maladaptive | 18.7 | 17.2 | −8.02 | p = 0.0090 |
| Behavior-Externalizing | (2.42) | (2.66) | | |

*P-values are presented for the comparison of the Week 12 value to the Baseline value, among those who completed the study (n = 18).

Among the 18 patients who completed 12 weeks of treatment, average improvement in overall anxiety and depression (ADAMS Total Score) reached 44% (p<0.01), with particular benefit observed for the General Anxiety (51%; p<0.01) and Compulsive Behavior subscales (48%; p<0.05). Additionally, improvements as measured by ABC_{FXS} ranging from 28% (Hyperactivity subscale; p0<0.05) to 60% (Stereotypy subscale; p0<0.01) were observed in aberrant behavior, with the Social Avoidance (p<0.01) and Social Unresponsiveness/Lethargy subscales (p<0.01) each improving by 55% during the treatment period. Beyond individual symptoms, quality of life improved by 17% (p=0.01).

The trial successfully met its primary endpoint, achieving a 44% improvement (P<0.0001) in the total ADAMS score at week twelve compared to baseline. The trial also achieved clinically meaningful improvements in all measures of the ABC-FXS, which address the key symptoms of FXS including irritability, hyperactivity, social unresponsiveness, social avoidance, stereotypy, and inappropriate speech.

Following the 12-week open-label study, patients were allowed to roll into a 1-year open-label extension study. 72% (n=13) of the 18 patients who completed the initial 12-week study rolled into the extension. While the open-label extension is ongoing, some data have been collected through Week 38 (12 weeks in initial study and up to 6 months in the extension study). Results from the extension study demonstrate continued gains in the two measures collected (ADAMS and ABC_{FXS}). Indeed, those who have completed a Week 38 visit (n=4) showed significant gains from screening in overall anxiety and depression, with participants experiencing an average improvement in the ADAMS total score of 74%. Similar improvement was observed for aberrant behavior, ranging from 75% (Irritability subscale) to 96% (Social Avoidance subscale) and 97% (Socially Unresponsiveness/Lethargy subscale) at Week 38.

The open-label extension continues to be ongoing and data has been collected through Week 51. The results are summarized in Table 8 (ABC_{FXS}) and Table (ADAMS).

TABLE 8
(ABC_{FXS})

| | | Week 12 | Week 38 | Week 51 | |
|---|---|---|---|---|---|
| | Screening | Mean | Mean | Mean | |
| | (baseline | Change | Change | Change | |
| | score) | (%) | (%) | (%) | Week 51 |
| | N = 12 | N = 12 | N = 9 | N = 9 | P values |
| Irritability | 22.3 | 51.1 | 63.7 | 59.2 | 0.0007 |
| Hyperactivity | 16.6 | 36.7 | 48.2 | 40.4 | 0.0037 |
| Socially | 10.8 | 65.7 | 83.3 | 72.2 | 0.0035 |
| Unresponsive/ | | | | | |
| Lethargic | | | | | |
| Social Avoidance | 5.7 | 57.9 | 75.4 | 77.2 | 0.0013 |
| Stereotypy | 9.7 | 60.8 | 73.2 | 64.9 | 0.0012 |
| Inappropriate | 6.2 | 56.5 | 66.1 | 56.5 | <0.0001 |
| Speech | | | | | |

TABLE 9
(ADAMS)

| | | Week 12 | Week 38 | Week 51 | |
|---|---|---|---|---|---|
| | Screening | Mean | Mean | Mean | |
| | (baseline | Change | Change | Change | |
| | score) | (%) | (%) | (%) | Week 51 |
| | N = 12 | N = 12 | N = 12 | N = 12 | P values |
| Manic/ | 8.8 | 34.1 | 53.4 | 45.5 | 0.0014 |
| Hyperactivity | | | | | |
| Depressed Mood | 3.2 | 43.8 | 62.5 | 59.4 | 0.0032 |
| Social Avoidance | 9.9 | 52.5 | 61.6 | 55.6 | 0.0004 |
| General Anxiety | 9.8 | 55.1 | 58.2 | 58.2 | <0.0001 |
| Compulsive | 3.2 | 50.0 | 59.4 | 59.4 | 0.0213 |
| Behavior | | | | | |
| Total Score | 33.3 | 48.6 | 59.2 | 54.4 | <0.0001 |

CBD gel was well tolerated, with excellent skin tolerability. One patient discontinued due to worsening of pre-existing eczema. No other adverse events led to discontinuation and no adverse events were considered severe. The most common adverse events were mild-moderate gastroenteritis (n=6) and upper respiratory tract infection (n=5). However, no patient experienced drug-related GI events during the 12-week treatment period and no THC was detected in the plasma.

The clinical results of the trial are significant for the many patients worldwide with FXS who currently have no approved therapeutic options to treat their symptoms. The data, in particular the improvements in anxiety, social avoidance, and irritability as measured by ADAMS, ABC-FXS and PARS-R, are significant. The CBD gel was very well tolerated in children and adolescents with FXS.

### Example 2: Patient Monograph as Reported by Parent

This is the report regarding a 7-year-old child participating in the above study and continuing on an extension study—as reported by the caregiver. The caregiver's son has full mutation Fragile X Syndrome. He is reported, prior to the trial, to be non-verbal, severely intellectually impaired, visually impaired, still in need of diapers and as having very severe GI issues requiring that he is fed by a feeding tube every two hours. Prior to the beginning the trial the child never ever made eye contact, rarely could leave his home without severe emotional distress, did not initiate any form of communication at all, intensely disliked being touched including by his parents, would not allow even family to sit next to him, and would leave the room if anyone walked into it.

Within the first two weeks of the trial, the patient began to make more eye contact, initiated physical contact with his family, e.g., grabbing his mother's hand, initiated emotional contact with his family including seeking to be in the same room with his family, and exhibited improved ability to leave the house, even to the extent the family could take their very first vacation together.

After the end of the initial trial and a few weeks into the extended trial, the caregiver recorded another big change in the patient. He started greeting his family, initiated and engaged in games that are more complex, exhibited/shared preferences for things instead of only rejecting all choices, and he began acknowledging the family pets. He also allowed his doctor to touch him and hold onto him without getting distressed. Patient began to use body signing (sign language) for the very first time. Patient communicated very clearly that he missed his mother for the very first time and was eager to be embraced and held by his mother.

Patient is reported to be happier, more relaxed, able to engage the world in ways he could not before, and able to learn new skills that he could not previously. His teachers, therapists and aids have also remarked in the changes in the patient.

### Example 3: Treatment of Subject with Full Methylation (Fmet) FXS with CBD

A 14-week pivotal Clinical study Of CaNNabidiol (CBD) in ChildrEn and AdolesCenTs with Fragile X (CONNECT-FX) trial was conducted. The randomized, double-blind, placebo-controlled CONNECT-FX trial assessed the efficacy and safety of Zygel CBD gel as a treatment for behavioral symptoms of FXS.

Two hundred and forty-five (245) patients with Fragile X syndrome, confirmed with the full mutation of the FMR1 gene, were enrolled at 21 clinical sites in the United States, Australia, and New Zealand. All patients were given placebo during the first two weeks (called a “placebo run-in” which is often used in neuropsychiatric clinical trials), and as a result 33 patients were not randomized. The remaining 212 patients were included in the Intent-to-Treat (ITT) population (Zygel: n=110; placebo: n=102) and were randomized to receive either trial drug or placebo for an additional 12 weeks. One patient did not receive study medication so 211 patients are included in the safety analysis (Zygel: n=109; placebo: n=102.) One patient did not have a post-baseline efficacy measure, resulting in 210 patients in the full analysis set (Zygel: n=109; placebo: n=101).

For the efficacy full analysis set, there were 210 patients enrolled (Zygel: n=109; placebo: n=101). A weight-based dose of Zygel, either 250 mg daily or 500 mg daily, was provided. The primary endpoint was social avoidance subscale of the ABD-C_{FXS}. Secondary endpoints included irritability subscale of the ABC-C_{FXS}, socially unresponsive/lethargic subscale of the ABC-C_{FXS}, and improvement in clinical global impression (CGI-I).

The Clinical Global Impression Scale of Improvement (CGI-I) is commonly used in clinical trials (Leigh et al. 2013) as it allows the clinician to utilize the history from the caregiver and incorporate the score into a clinical rating for the severity of symptoms. CGI-I is a 7-point scale that requires the clinician to assess how much the patient's illness has improved or worsened relative to a Baseline state at the beginning of the intervention and rated as: 1—very much improved; 2—much improved; 3—minimally improved; 4—no change; 5—minimally worse; 6—much worse; or 7—very much worse. Information from both the clinician and the parent/caregiver history are incorporated into a clinical rating.

Patients with FXS have a full mutation of FMR1 gene and can be described as: (i) Non-Mosaics (patient has all cells with full mutation of FMR1 gene and all genes are fully methylated; note—methylation inactivates the FMR1 gene); (ii) Size mosaicism (patient has some cells with full mutation of FMR1 gene and some cells with pre-mutation of the FMR1 gene and not all genes are methylated); and (iii) Methylation mosaicism (all cells have full mutation of FMR1 gene but not all genes are methylated).

Patients with >90% methylation are considered to have a fully methylated FMR1 gene and do not produce mRNA and therefore no FMR protein. Analysis was planned to explore differences in two groups of patients—with full methylation and without full methylation.

Results

Table 10 summarizes the results of CONNECT-FX in the full analysis population for the primary and key secondary endpoints.

TABLE 10
Full Population Analysis

| | Placebo | | Zygel | | | |
|---|---|---|---|---|---|---|
| | N = 101 | | N = 109 | | | |
| | | Week 12 | | Week 12 | | |
| | Baseline | Mean | Baseline | Mean | Treatment | Treatment |
| Endpoint | Mean | Change | Mean | Change | Difference* | P-Value |
| ABC-C_{FXS} | 7.24 | −2.29 | 7.12 | −2.68 | −0.39 | NS |
| Social Avoidance | | | | | | |
| Subscale | | | | | | |
| ABC-C_{FXS} | 27.65 | −4.14 | 28.49 | −5.88 | −1.74 | NS |
| Irritability Subscale | | | | | | |
| ABC-C_{FXS} | 12.82 | −3.14 | 13.42 | −3.50 | −0.36 | NS |
| Socially Unresponsive/ | | | | | | |
| Lethargy Subscale | | | | | | |
| CGI-I at week 12 | — | 15.9% | — | 20.2% | 1.33** | NS |
| (Much and Very | | | | | | |
| Much Improved) | | | | | | |

NS = not statistically significant
*A negative treatment difference demonstrates Zygel patients improved versus placebo
**Odds Ratio

Zygel did not achieve statistical significance versus placebo in the primary endpoint of improvement in the Social Avoidance subscale of the Aberrant Behavior Checklist—Community FXS (ABC-CFXS). Zygel also did not demonstrate statistical significance versus placebo in the three key secondary endpoints, which were the change from baseline to the end of the treatment period in the Irritability subscale score of the ABC-CFXS, the Socially Unresponsive/Lethargic subscale score of the ABC-CFXS and Improvement in Clinical Global Impression (CGI-I).

An analysis of the most severely impacted patients in the trial, as defined by patients having at least 90% methylation (“full methylation”) of the impacted FMR1 gene, demonstrated that patients receiving Zygel achieved statistical significance in the primary endpoint of improvement at 12 weeks of treatment in the Social Avoidance subscale of the ABC-C_{FXS} compared to placebo (p=0.020). This group comprised 80% of the patients enrolled in the CONNECT-FX study, and it is believed that full methylation occurs in approximately 60% of the overall FXS patient population. The terms “Fragile X Syndrome in a subject having full methylation of the impacted FMR1 gene” refers to subjects having “Fmet Fragile X Syndrome” or “Fmet FXS.” Patients with genetically confirmed full mutation Fragile X and full methylation of their impacted FMR1 gene are generally the most severely impacted by the disorder. Within the CONNECT-FX trial, this was corroborated with patients in the analysis at baseline having higher anxiety, lower IQ, lower adaptive function, and more severe autism as compared to patients without a fully methylated FMR1 gene.

One hundred and sixty-nine (169) patients met the criterion of full methylation of the impacted FMR1 gene. One patient was not treated and one did not have a post-baseline efficacy measure, resulting in 167 patients (Zygel: n=91; placebo: n=76). Table 11 summarizes the patient data for full methylation (>90% methylation, Fmet) versus not fully methylated (<90% methylation, Not-Fmet).

TABLE 11
Full Methylation (Fmet) FXS Subpopulation

| | Full Data | Fmet | Not-Fmet |
|---|---|---|---|
| Patients | Set | (≥90%) | (<90%) |
| Full Analysis Set | 210 | 167 | 42 |

Table 12 summarizes results of CONNECT-FX in the analysis set of patients with full methylation of the impacted FMR1 gene across the primary and key secondary endpoints.

TABLE 12
Full Methylation (Fmet) Subpopulation Analysis

| | Placebo | | Zygel | | | | |
|---|---|---|---|---|---|---|---|
| | N = 76 | | N = 91 | | | | |
| | | Week 12 | | Week 12 | | | |
| | Baseline | Mean | Baseline | Mean | Treatment | Odds | Treatment |
| Endpoint | Mean | Change | Mean | Change | Difference** | Ratio | P-Value |
| ABC-C_{FXS} | 7.18 | −1.99 | 7.12 | −2.99 | −1.0 | | 0.020* |
| Social Avoidance | | | | | | | |
| Subscale | | | | | | | |
| ABC-C_{FXS} | 28.0 | −4.13 | 29.36 | −6.43 | −2.30 | | 0.091 |
| Irritability Subscale | | | | | | | |
| ABC-C_{FXS} | 13.17 | −2.74 | 13.30 | −3.91 | −1.17 | | 0.135 |
| Socially Unresponsive/ | | | | | | | |
| Lethargy Subscale | | | | | | | |
| CGI-I at Week 12 | — | 35.7% | — | 51.1% | | 1.88 | 0.056 |
| (Any Improved) | | | | | | | |

*statistically significant vs. placebo
**A negative treatment difference demonstrates Zygel patients improved versus placebo

The median improvement in the Social Avoidance subscale of the ABC-CFXS after twelve weeks of treatment was 40% for patients on Zygel and 21.1% for patients on placebo.

The interaction test of heterogeneity for the Social Avoidance subscale was statistically significant (p=0.002), which means that the difference in treatment effects between the subgroups was statistically significant.

### Safety Data

Zygel was very well tolerated in CONNECT-FX, and the safety profile was consistent with previously released data from other Zygel clinical trials. No safety signal was identified. Approximately half (54%) of the 211 patients included in the safety population experienced a treatment emergent adverse event (any event, whether unrelated or related to study drug), all of which were mild or moderate. The frequency of treatment emergent adverse events was similar across treatment groups (58% of patients on Zygel, 50% of patients on placebo). There were no serious or severe adverse events reported during the study. There were seven total psychiatric disorder TEAEs, five of which were in the placebo group.

Only 15 (7%) patients experienced a treatment-related adverse event (20 events total); 11 patients on Zygel experienced 14 treatment related TEAEs, while four patients on placebo experienced six TEAEs. The most common treatment-related TEAE was application site pain (Zygel: 6.4%; placebo: 1.0%).

Laboratory values for chemistry and hematology were comparable between the placebo and Zygel treatment groups, and there were no clinically relevant abnormalities in either group. Specifically, there were no elevated liver function tests (LFT) in the Zygel group, and two modestly elevated LFTs in the placebo group.

This study identified an important population of patients who appear to benefit significantly from treatment of their behavioral symptoms of FXS with Zygel. When combined with the excellent tolerability seen in this trial, it is believed Zygel could be an important therapeutic option for the most severely impacted patients with Fragile X Syndrome.

1. A method of treating one or more symptoms of Fragile X Syndrome in a subject having full methylation of the FMR1 gene, the method comprising:
transdermally administering an effective amount of cannabidiol (CBD) to the subject.

2. The method according to claim 1, wherein one or more symptoms is a behavioral symptom of Fragile X Syndrome.

3. The method of claim 2, wherein the behavioral symptom is social avoidance.

4. The method according to claim 1, wherein treating includes improvement in clinical global impression (CGI-I).

5. The method according to claim 1, wherein the total daily dose of CBD is between about 50 mg and about 500 mg total daily.

6. The method according to claim 1, wherein the total daily dose of CBD is 250 mg.

7. The method according to claim 1, wherein the total daily dose of CBD is 500 mg.

8. The method according to claim 1, wherein the CBD is formulated as a gel.

9. The method according to claim 1, wherein the CBD is administered in a single daily dose.

10. The method according to claim 1, wherein the CBD is administered in two daily doses.

11. The method according to claim 1, wherein the CBD is transdermally administered on the subject's arm.

12. The method according to claim 1, wherein the CBD is a synthetic CBD.

13. The method according to claim 1, wherein the CBD is botanically derived.

14. The method according to claim 1, wherein transdermally administering an effective amount of CBD reduces an intensity of at least one adverse event relative to orally administering CBD.

15. The method according to claim 1, wherein the at least one adverse event is selected from the group consisting of somnolence, psychoactive effects, liver function, and GI related adverse events.
